(19) United States
(12) Patent Application Publication
(10) Pub. No.: US 2026/0345292 A1
(43) Pub. Date: Nov. 5, 2026

(54) ROOT TIP GUIDE

(71) Applicant: King Abdulaziz University, Jeddah (SA)

(72) Inventors: Rawan Eskandar, Jeddah (SA); Mohammed Howait, Jeddah (SA)

(21) Appl. No.: 19/198,865

(22) Filed: May 5, 2025

(51) Int. Cl. A61C 1/08; A61B 6/51

(52) U.S. Cl. CPC A61C 1/082; A61B 6/51

(57) ABSTRACT

One example device includes a support block, having a biting surface extending in a plane, an opposing biting surface, and is manually placeable into and positionable within an oral cavity to align with a subject tooth. In the alignment the biting surface extends parallel to and contacts the occlusal surface of the subject tooth. A positionable visible guide includes a pivot coupler with one member fixed to the support block in alignment with a pivot axis, and engaged with another member at a selectable rotation about the pivot axis. The positionable visible guide includes an arm, having a coupling end fixed to the pivot coupler. Attached to the arm is a visible target position indicator spaced a distance along a displacement axis from the coupling end. The displacement axis intersects the reference plane at an angle, and the pivot coupler is configured such that the angle is selectable.

### BACKGROUND

### Technical Field

The present disclosure generally relates to three dimensional (3D) guides for endodontic microsurgery (EMS) and more particularly to 3D root tip guides for EMS surgical instruments.

### Description of Related Art

In various EMS root tip procedures, it can be desired that the surgical instrument's operative surface be aligned to the root tip to avoid damaging surrounding tissues, e.g., the inferior alveolar nerve (IAN), and maxillary sinus. One conventional EMS technique directed to such alignment is referred to as “3D-guided” EMS. In this technique, a scan of the patient's jaw is obtained, e.g., by cone-beam computed tomography (CBCT) to identify the position of the root tip. Using software algorithms linked with a particular 3D guide template, the CBCT scan data is used to shape and arrange the template's surrounding tooth support engagement surfaces and instrument guide surface(s) in a patient-specific configuration. A patient-specific 3D guide is then fabricated, via 3D printing, The resulting structure can be arranged in the patient's oral cavity to function as a guide for the EMS root tip procedure.

This technique, though, has multiple shortcomings. One is requirement of a high-resolution intraoral scanner, particular 3D planning software, and a high quality 3D printer. Such resources are not always available in routine clinical practice. Another shortcoming that it can require multiple, time consuming steps, including multiple visits by the patient. Another is sterilization concerns. 3D printed templates are compatible with autoclave sterilization; specific and costly material like Surgical Guide Resin can be required;

For the reasons above, new and improved techniques of root tip guides are needed.

### SUMMARY

This Summary presents example features and aspects and is not an exclusive or exhaustive description of disclosed subject matter, and whether or not a feature is included in this Summary is not intended as any indication of the feature's relative importance or benefit.

One example visible target position reference device according to one or more embodiments may include a support block, and the support block may be configured with an external shape geometry that can include a first biting surface that can extend in a plane and a second biting surface that can be arranged opposite the first biting surface. The external shape external shape geometry can be further configured, according to one or more embodiments, such that the support block can be manually placeable into and positionable within a human oral cavity into an alignment with a subject tooth comprising the first biting surface extending parallel to the occlusal plane of and contacting the occlusal surface of the subject tooth. Configurations of the device may further include a selectively positionable visible guide member. The selectively positionable visible guide member may further include a pivot coupler having a first pivot member that can be fixed to the support block in alignment with a pivot axis, and a second pivot member that can be engaged with the first pivot member at a selectable rotation angle about the pivot axis. The selectively positionable visible guide member may include a pivot coupler that may include a support arm, which may have a coupling end fixed to the second pivot member. The selectively positionable visible guide member may further include, according to one or more embodiments, a visible target position indicator that may be spaced a distance along a displacement axis from the coupling end, and that can be configured such that the displacement axis intersects the reference plane at an angle. Further, the pivot coupler may be further configured such that the angle is selectable.

One method for guided endodontic root tip surgery, in accordance with various embodiments, may include a step of applying a cone-beam computed tomography (CBCT) measuring to a patient, outputting a root tip length data and a root angulation data, and applying a step, based at least in part on the root tip length data and the root tip angulation data, a constructing a configured visible target position reference device according to one or more embodiments, wherein the angle with the reference plane can be based at least in part on the root tip angulation data.

### BRIEF DESCRIPTION OF DRAWINGS

A further understanding of the nature and advantages of the embodiments may be realized by reference to the following drawings. It will be understood that the drawings show illustrative, not limitative features and examples. It will also be understood that the drawing graphics are scaled for readability and that graphic representations of functions and features are not necessarily drawn to a scale consistent with their physical implementations. Further, in the drawings similar components or features may have the same reference label.

FIG. 1a shows a front view; FIG. 1b a bottom view; and FIG. 1c a side view of one example root tip surgery guide device according to one or more embodiments, presented with the example's selectively positionable target position guide set in one example target position state selected arbitrarily, for illustration, from an optionally configurable range of and/or from among an optionally configurable plurality of different target position states.

FIG. 2 shows a side view of the FIGS. 1a, 1b, and 1c example root tip surgery guide device set in another example target position state selected arbitrarily, as another illustration, from the optionally configurable range of and/or from the optionally configurable plurality of different field settable target position states.

FIG. 3 shows a side view of the FIGS. 1a, 1b, and 1c example root tip surgery guide device set in still another example target position state selected arbitrarily, as still another illustration, from the optionally configurable range of and/or from the optionally configurable plurality of different field settable target position states.

FIG. 4a shows a front view of another example root tip surgery guide device according to another one or more embodiments, further comprising one example ruler-type arm extension indicator, and shown as set in one example target position state; FIG. 4b shows the example root tip surgery guide device of FIG. 4a but set in another example target position state, by a rotation of the ruler-equipped extendible arm to another angulation position; and FIG. 4c shows the example root tip surgery guide device of FIG. 4a set in still another example target position state, by a retraction of the ruler-equipped extendible arm to another root tip length.

FIG. 5 shows one captured CBCT scan image of a root tip region and proximal tissue region of one subject, labelled with root tip position and root angulation axes, which are referenced herein in further detailed description of example steps and acts operations of and aspects in preoperative setting of and in-situ of root tip surgical guide devices according to one or more embodiments.

FIG. 6 shows a rendered outline of one arbitrarily selected mandibular molar, from an CBCT scan image of an example subject, annotated to show another example of root tip position and root angulation information usable in configuring of root tip surgical guide devices according to one or more embodiments.

FIG. 7 shows a side view of the FIGS. 1a, 1b, and 1c implementation of one root tip surgical guide device according to one or more embodiments, after setting via configuration of guide extension-angulation, into a target position stated aligned to the FIG. 6 captured root tip position angulation parameters.

FIG. 8 shows a side view of the FIG. 7 guide extension-angulation state configuration of the FIGS. 1a, 1b, and 1c root tip surgical guide device according to one or more embodiments, rendered in an illustrative bite clench securement between the occlusal surface of the FIG. 7 subject mandibular molar and the occlusal surface of a representative opposing molar, further showing registration of the opposing molar occlusal surface.

FIG. 9 shows a partial 3D perspective rendering of the FIG. 8 side view, showing further aspects of the bite clench securement.

FIG. 10a shows a front view; and FIG. 10b shows a side view, on the FIG. 10a projection plane 10b-10b, of one example implementation of a position pointing tip configured one root tip surgical guide device according to another one or more embodiments, and set in one example root tip extension-angulation state.

FIGS. 11a, 11b, and 11c show 3D perspective views, from the same view perspective, of three different example root tip extension-angulation configuration states of another root tip surgical guide device according to one or more embodiments, wherein FIG. 11a shows one alternative extendible support arm structure in one example extension state while, concurrently, in one cooperative engagement rotation state; FIG. 11b shows the FIG. 11a root tip surgical guide device with its alternative extendible support arm retracted to another extension state and, concurrently, in the same cooperative engagement rotation state; and FIG. 11c shows the FIG. 11a root tip surgical guide device with its alternative extendible arm structure in the FIG. 11b extension state and, concurrently, supported in another cooperative engagement rotation state.

### DETAILED DESCRIPTION

The term “subject tooth” as used herein, references a tooth of a patient having a respective root that is the target of an EMS procedure. The subject tooth can be describable for example and without limitation, as an “offending tooth,” an “afflicted tooth,” an “impaired tooth,” and/or a “diseased tooth.”

The term “opposing tooth,” as used herein, references a tooth of a patent that, when the patient brings the patient's teeth into a state of occlusion, has occlusal surfaces that contact and align with the occlusal surfaces of the subject tooth.

The term “occlusal plane,” as used herein in reference to or other association with a particular example subject tooth, or the subject tooth's opposing tooth means a reference-type plane that is defined by the occlusal peaks or cusps of the subject tooth or the occlusal peaks or cusps of the subject tooth's opposing tooth.

The meaning of “any selected subject tooth of a patient,” as used herein in the context of describing structure of embodiments and/or use of such embodiments in relation to a patient, can encompass “any selected subject tooth that is within the application range of the particular implementation and specification of the bite-grip secured root tip EMS guide device being used.” For example, persons of ordinary skill the art(s) relating to this disclosure will understand, after reading this disclosure, that because of the dental anatomy variations among dental patients it may be desired to include in preop procedures a selecting of bite-grip secured EMS guide devices from among different sets based, for example, on patient age, gender, and by the particular tooth that is the subject tooth. One implementation, for example and without limitation, can comprise producing N different shape-dimension classes or types of bite-grip secured root tip EMS guide devices according to one or more embodiments. Each of the shape or dimension types or classes may be usable for teeth of dimensions within a class-specific type-specific range. An EMS service facility may maintain, for example, an inventory of integer A1 “type 1” or “class 1” dimension root tip EMS guide devices and integer A2 “type 2” or “class 2” imension root tip EMS guide devices and so forth.

Structure in implementations of occlusal force secured, i.e., bite-grip secured root tip EMS guide devices according to one or more embodiments can include a bite-clenchable alignment and support block and, supported in a positionable and, in some embodiments, repositionable manner by the alignment and support block, a target position visible indicator device. According to various embodiments, the bite clenchability functionality of the bite-clenchable support block can be provided by configuring the block, with respect to external dimensions and geometry, to be manually placeable within a patient's mouth and manually positionable into an alignment state. The alignment state and corresponding configuration of the alignment and support block can be such that, in the alignment state, the first external surface faces and is an lateral registration with the occlusal surface of the subject tooth. In addition, according to various embodiments, the configuration of the support block can be such that when the block is positioned in the above-described alignment state, the second external surface faces toward the occlusal surface of the opposing tooth. The configuration, with respect to “faces toward the occlusal surface of the opposing tooth” can be such that a result of a bite clenching of the bite-clenchable support block by a patient while the block is in the alignment state can include the first external surface being in registration with and receiving contact and occlusal force from the occlusal surface of the subject tooth, concurrent with the second external surface being in registration with and receiving contact and occlusal force from the occlusal surface of the opposing tooth.

According to various embodiments the first external surface may be arranged such that its functionality includes providing a reference surface, and further to that functionality the first external surface may have a planar configuration, at least over an area sufficient to fully accommodate the occlusal surface of the subject tooth. Features of various configurations and arrangements of bite-grip secured root tip EMS guide devices include structures configured to a position according to various embodiments, Functionalities of this surface, as described in more detail in later sections of this disclosure, includes capability of aligning with the occlusal plane of the subject tooth.

One alternative can include placing a cushion under and over the support block. A result, though, can be positioning the oscular surface of the subject tooth above or beneath the plane of the first biting surface, which is also the plane for the pivot axis. Therefore, positioning the target point a distance and rotational angle from an axis aligned with the first biting surface will not coincide with a position that is with reference to the oscular plane of the subject tooth.

One solution can include an offset mount that positions the pivot axis at an offset below the reference surface, the offset being equal to the cushion thickness.

Another solution can be to adjust the displacement length and the rotation angle to compensate for the offset position of the pivot axis.

According to some embodiments, the target position indicator may be configured to have functionality comprising, e.g., visible pointing. The target position indicator, according to some other embodiments, may be configured to provide via, e.g., and without limitation, a ring-shaped body, combination functionality as a visible position indicator and a mechanical guide for EMS instruments such as, without limitation, an EMS curette or an EMS drill.

According to various embodiments, the first external surface, i.e., the fit biting surface can be configured as planar, extending within a plane that for convenience in subsequent description of structures and of example applications and operations will be alternatively referred to as a “reference plane.”

According to various embodiments, the selectable extension-selectable angulation support structure can include a support arm having a first end a pivot coupling mechanism having a first pivot member that is fixed to the bite plate in alignment with a pivot axis, and a second pivot member that is engaged with the first pivot member at a selectable rotation angle, about the pivot axis. There may be, mechanically coupled to the bite-clenchable guide support block, a selectable extension-selectable angulation support structure and attached to the selectable extension selectable angulation support structure.

It will be understood that “visible,” as used herein in the context of a visible pointer to or visible indicator of a target position, means optically detectable by a surgeon on a surface of the patient's jawbone, the surface being an area on the jawbone that becomes visible after soft tissues, e.g., gums or mucosal tissue, are reflected or moved away during surgical procedure.

A shared functionality of the first bite surface and the second bite surface, in bite-grip secured root tip EMS guide devices according to various embodiments, include enabling patient actuatable, patient releasable securing, during surgery of a guide device that inherently aligns its reference plane to the occlusal plane of the subject tooth.

Accordingly, the term “first bite surface” is used herein to refer to the bite surface that will face and will receive bite force from the occlusal surfaces of the subject tooth, and the term “second bite surface” is used to refer to the bite surface of the bite plate that will face and receive bite force from the opposite tooth. This naming assignment is arbitrary, but the assigned naming is used consistently in this description.

According to some embodiments the selectively positionable indicator support structure can be configured to couple to and support the EMS target position visible indicator at positions with a range of positions along an arcuate trajectory that can be centered, for example, about a pivot axis. The selectively positionable indicator support structure can be further configured, according to some embodiments, to couple to and support the EMS target position visible indicator at positions spaced apart in a radial direction toward and/or away from the pivot axis. The selectively positionable indicator support structure can be additionally configured, according to some embodiments, to be capable of supporting of and of fixing of the EMS target position visible indicator at selectable positions along the arcuate trajectory centered about the pivot axis, as well as supporting of and fixing of the EMS target position visible indicator at selectable positions in the radial direction, toward and/or away from the pivot axis.

According to some embodiments the selectively positionable indicator support structure can be configured to be capable of supporting of and of fixing of the EMS target position visible indicator at selectable positions along the arcuate trajectory, and at selectable positions along the radial direction, can be further configured such that the arcuate trajectory and the selectable positions can be constrained within a target position plane.

According to some embodiments the selectively positionable indicator support structure can comprise a support arm that has a coupling end and, in combination with the support arm, a coupling mechanism configured to couple the coupling end to, for example, a side of the bite plate. According to one or more embodiments the side of the bite plate can be a side that will be visible, for example, by a surgeon, when the bite plate is arranged in a patient's mouth with the first bite surface facing the occlusive surface of the subject tooth. According to one or more embodiments, the visible target position indicator can be attached to the support arm, at a distance from the coupling end. The coupling mechanism can be configured to rotatably couple the coupling end, such that the visible target position can be moved along an arcuate trajectory can be rotated, about a pivot axis, between different rotational positions.

According to one or more embodiments, the coupling mechanism can be further configured such the arcuate movement, at least over an arcuate movement ranges, is constrained with a position plane. The coupling mechanism can be additionally configured such that the position plane can extend normal to the first bite surface.

### Example One—Arm Pivotabky Coupled-Ring Target Guide

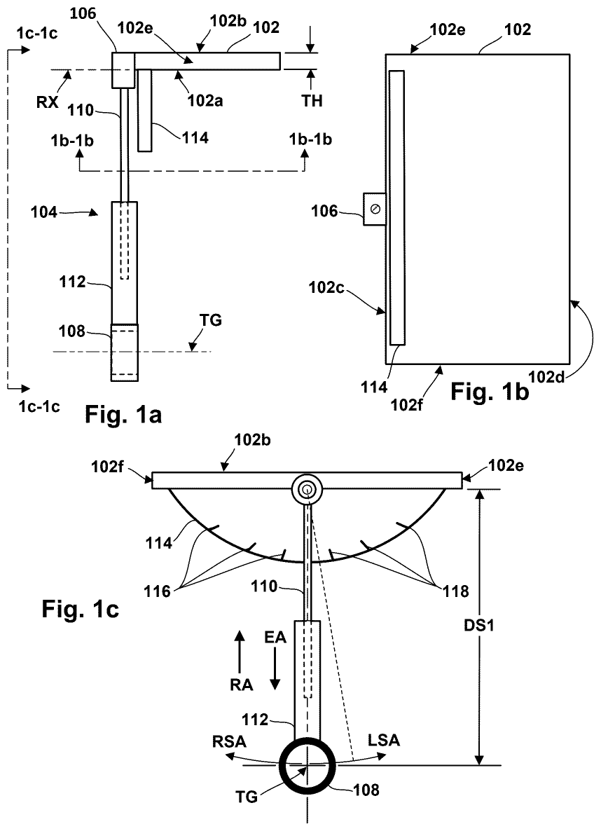

FIG. 1a shows a front view of one example implementation 100 of one root tip surgical guide device (referenced hereinafter as “root tip guide 100”) according to one or more embodiments. FIG. 1b shows a bottom view of the root tip guide 100, as viewed on FIG. 1a projection plane 1b-1b, and FIG. 1c shows a side view of the root tip guide 100, as viewed on FIG. 1a projection plane 1c-1c,

The FIG. 1a root tip guide 100 includes a support block 102, having a first bite reception surface 102a spaced by thickness TH from a second bite reception surface 102b. The support block 102 can be configured such that the first bite reception surface 102a can be positioned in an alignment position facing the occlusal surface of a subject tooth, e.g., an afflicted molar of a patient. The second bite reception surface 102b can be configured to face opposite the first bite reception surface 102a, such that, in the alignment position, the second bite reception surface 102b faces the occlusal surface of, e.g., an opposing molar. As will be understood from reading this disclosure in its entirety, for use with mandibular molars the support block 102 is arranged above the afflicted molar, with the first bite surface 102a facing downward, toward and contacting the afflicted molar occlusal surface. For use with afflicted maxillary molars the support block 102 can be aligned under the occlusal surface of that maxillary molar, with the first bite surface 102a facing upward.

The FIG. 1a-1c root tip guide 100 can further include an extendible arm 104 that can connect at a first end to a coupler 106, to be rotatably coupled to the support block 102. The coupler 106 can be configured, for example, with coupler portion that connects, e.g., is secured to the first end of the extendible arm 104 and a coupler portion that connects, e.g., is secured to an edge of the support block 102, where the portion that is secured to the first end of the extendible arm 104 can rotate about the portion secured to the support block, the rotation being about a pivot axis RX. The pivot axis RX, according to various embodiments, can be aligned to extend within the plane of the first biting surface 102a. Attached to, or structurally integrated with the extendible arm 104, can be a visible guide structure 108. In the FIGS. 1a, 1b, 1c example the visible guide structure 108 is configured as a guide ring having a geometric center, labeled “TG,” that is the target position, The guide ring implementation of the visible guide structure 108 can function as a visible target position indicator, i.e., the target position is in the middle of the circular ring. The guide ring implementation can further function, for example, as a positioning guide for a surgical instrument, such as but not limited to, an EMS drill.

As shown in the example implementation visible in FIGS. 1a-1c, the extendible arm 104 can be configured, for example, as a two element telescoping arm, comprising a fixed section 110 and extendible section 112, with “fixed” meaning no linear movement along the extension axis due to the coupler 106.

The FIG. 1a-1c root tip guide 100 can further include a protractor-type angulation indicator 114. According to some embodiments the protractor-type angulation indicator 114 can be configured with a functionality of being a visible reference for the rotation or angulation state of the extendable arm 104. Referring to FIG. 1c, an example implementation can include a marker to which the extendible arm 104 aligns when perpendicular to the first biting surface 102a and with three angulation position markers 116 to the left and three angulation position markers 118 to the right. Referring to FIG. 1c, it will be understood that viewed from the FIG. 1c projection the angulation position marker to which the extendible arm 104 aligns when perpendicular to the first biting surface 102a is concealed. The three angulation position markers 118 to the right of the center marker can be usable with the bite clenchable support block 102 clenched such that the fourth lateral edge 102f, which is the leftmost edge on FIG. 1c. faces and is adjacent to the patient's lower front teeth and the image plane of the figure faces the left cheek of the subject. This could be employed, for example, for EMS on the subject's left-side mandibular molars. For use with the subject's left side maxillary molars, the device 102 may be inverted, such that the first biting surface 102a faces upwards, such the third lateral edge 102e, which is the rightmost edge on FIG. 1c faces and is adjacent to the patient's upper front teeth and the guide member 108 is spaced above the first biting surface. This could be employed, for example, for EMS on the subject's left-side mandibular molars. For use with the subject's right side mandibular molars the device 100 may be reversed horizontally, such that the third lateral edge 102e faces toward the front of the patient's mouth, and the and such that first biting surface 102a faces downward, and the guide member 108 is spaced below the first biting surface. In this orientation the second lateral surface 102c faces in the direction of the patient's right cheek.

According to some embodiments the protractor-type angulation indicator 114 can be further structured, and in some embodiments the extendible arm 104 may be correspondingly further configured such that structure of the protractor-type angulation indicator can have certain contact with the extendible arm 104, in a manner to support the arm 104 against deflection to the right of the figure sheet due to left-to-right direction pressure applied, e.g., by the EMS surgeon. According to further embodiments, the protractor-type angulation indicator 114 can be further structured, and the extendable arm 104 correspondingly and cooperative further configured such that at a plurality of the angular positions a cooperative engagement between the extendable arm 104 and the structure 114 can provide a securement of the arm 104 in that position.

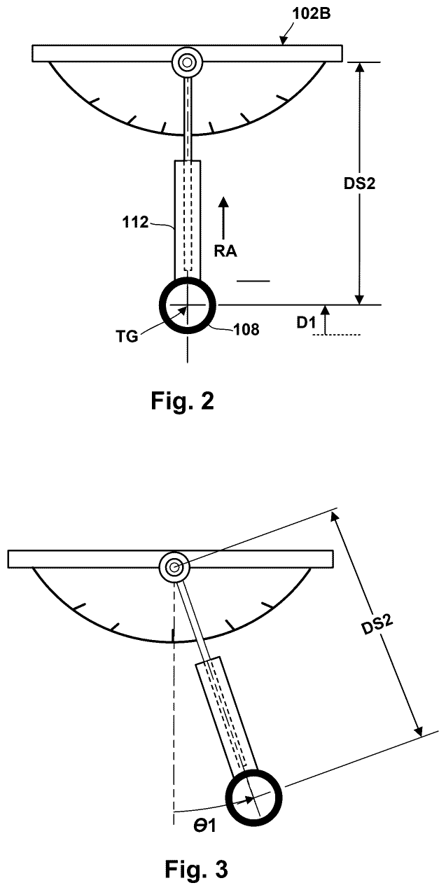

FIG. 2 shows a side view of the FIGS. 1a, 1b, 1c root tip guide 100, viewed on the same FIG. 1a projection plane 1c-1c, after a re-positioning of the extendable segment 112 by a retraction distance D1 in the retraction direction RA. The resulting displacement distance from the first bite reception surface 102a to the target position TG is DS2, which is D1 less than DS1. The angulation is unchanged.

FIG. 3 shows a side view of the FIGS. 1a, 1b, 1c root tip guide 100, viewed on the same FIG. 1a projection plane 1c-1c, after a re-positioning of the extendible segment 112 by a retraction distance D1 in the retraction direction RA. The resulting displacement distance from the first bite reception surface 102a to the target position TG is DS2, which is D1 less than DS1. The angulation is unchanged.

### Example Two—Ruler-Type Arm Extension Indicator

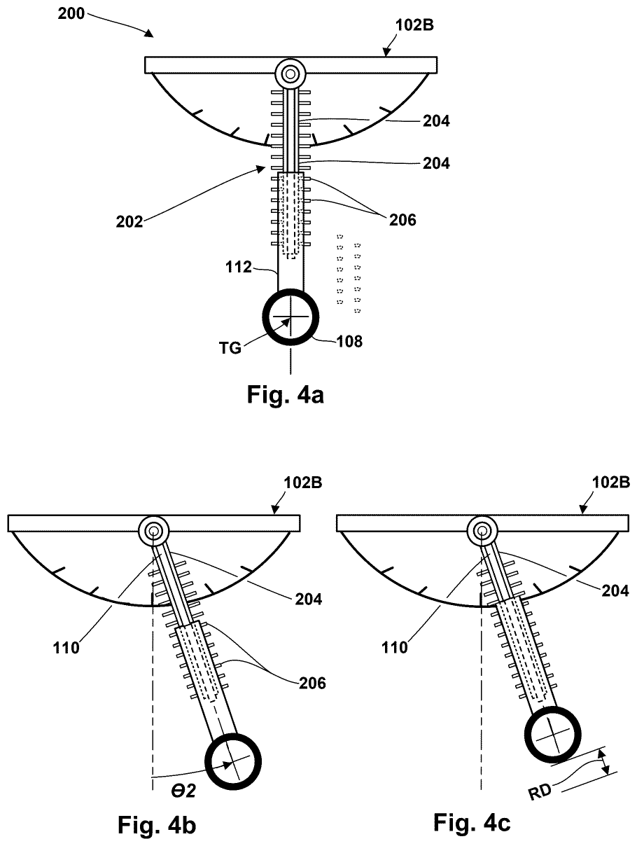

FIG. 4a shows a front view of one example root tip surgery guide device 200 according to another one or more embodiments, comprising the FIG. 1a-1c support block 102, extendible arm 104 pivotally coupled by the coupler 106 to the first lateral edge 102c, and further comprising a ruler-type arm extension indicator 202. The ruler-type extension indicator 202 can be implemented, as shown with a ruler support 204 and attached to or, for example, formed integrally with the ruler support 204, a plurality extension position markers 206. The extension position markers 206 are visible in FIGS. 4a-4c as equally spaced from one another, but this is not a limitation. and shown as set in one example target position state. FIG. 4b shows the example root tip surgery guide device 200 of FIG. 4a but set in another example target position state, by a rotation θ2 of the extendible arm 114 and its ruler-type arm extension indicator 20 to another angulation position. FIG. 4c shows the example root tip surgery guide device 200 of FIG. 4a set in still another example target position state, by a retraction of distance RD of the ruler indicator 202 equipped extendible arm 114 to another root tip length.

### Example Three—Preoperative and Intraoperative

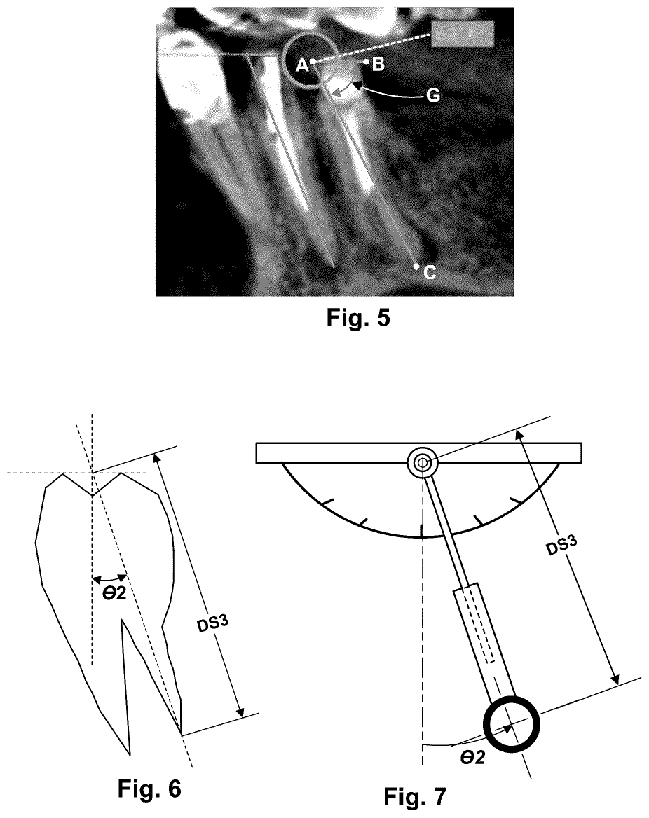

FIG. 5 shows one captured CBCT scan image of a root tip region and proximal tissue region of one subject, labelled with afflicted tooth occlusal point “A,” afflicted tooth other occlusal point “B,” and root tip position “C,” as well as reference line AB extending parallel to the occlusal plane of the afflicted tooth, and line segment AC that extends, from the other occlusal point B along the long axis of the root to the root tip A. Also labeled on FIG. 5 is the intersection angle “G,” which is the root angulation relative to the occlusal plane.

Referring to FIGS. 1a-1c it will be understood that the root tip position information obtained from the figure cam be used to configured to the root tip guide 100 to be used in EMS procedures on the root tip of the FIG. 5 tooth. More assuming the FIG. 1a-1c implementation incudes, for example, a threaded screw aligned with the pivot axis RX, such a screw may be loosened and the extendible arm then rotated to a rotation position corresponding to the angulation information obtained from the figure. Next, or first if desired, the extendible arm 104 may be retracted.

Stated more formally, there may a step one or first step of performing a scan of the patient's oral cavity. The scan may be, but is not limited to being CBCT. Nest there may be a step two or second step of obtaining from the scan the root length and angulation. Root length may be measured from a fixed reference point on the occlusal plane to the root apex of the affected tooth. Root angulation-determined by the angle between two lines, i.e., line A-B and line A-C. Wherein line A-B is parallel to the occlusal plane of the subject tooth, line A-C is drawn from the same fixed occlusal point toward the long axis of the root, and the intersecting angle (∠G) between these lines represents the root's angulation relative to the occlusal plane. Next, step three can comprise configuring a root tip guide using the scan image derived root length and root angulation data.

The practitioner may then, for example, place protective gloves on the practitioner's hand, place the configured root tip guide 100 or a configured version of another implementation, into the subject patient's mouth, align the device as described above, and may then instruct the patient to bite down on the configured device.

### Example Four—Another Preoperative and Intraoperative Procedure

FIG. 6 shows a rendered outline of one mandibular molar, from a CBCT scan image, annotated to show another example of root tip position and root angulation information usable in configuring of root tip surgical guide devices according to one or more embodiments.

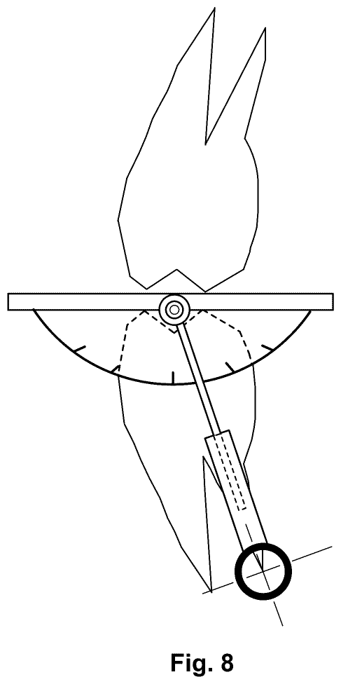

FIG. 7 shows a side view of the FIGS. 1a, 1b, and 1c implementation of one root tip surgical guide device according to one or more embodiments, after configuration into a guide extension-angulation state aligned to the FIG. 6 captured root tip position-angulation parameters.

FIG. 8 shows a side view of the FIG. 7 guide extension-angulation state configuration of the FIGS. 1a, 1b, and 1c implementation, in an illustrative bite clench securement between occlusal cusps of a representative subject molar and occlusal cusps of a representative opposing molar, with corresponding embodiment-provided alignment of the visible target position indicator to the representative subject molar root tip.

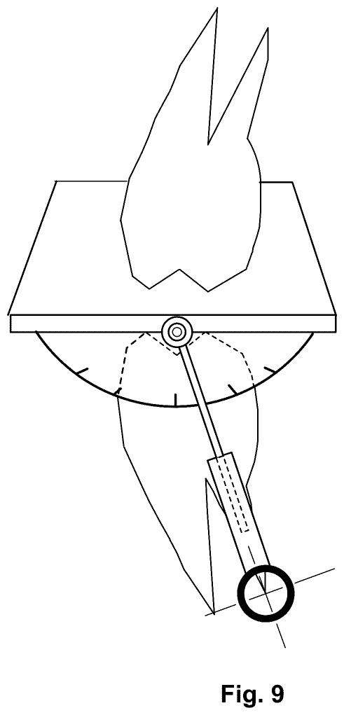

FIG. 9 shows a partial 3D perspective rendering of the FIG. 9 side view, showing further aspects of the bite clench securement.

### Example Five—Pivoting Arm—Pointer Type Target Guide

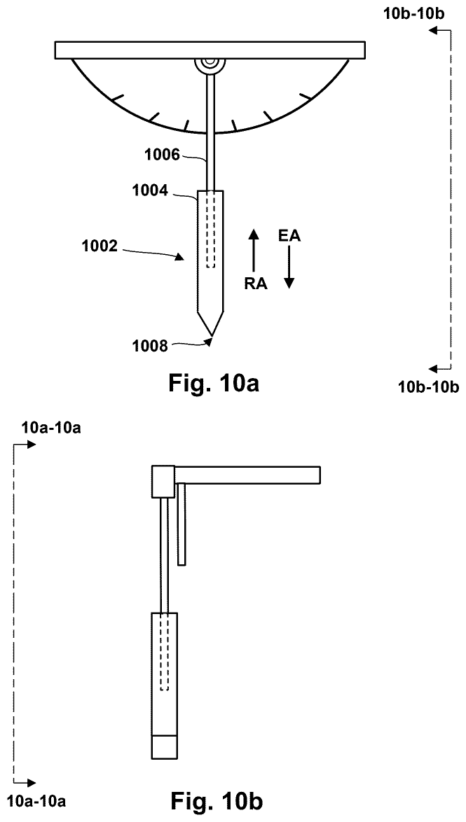

FIG. 10a shows a front view of one example implementation of a position pointing tip configured root tip surgical guide device 1000 according to another one or more embodiments, set in one example root tip extension-angulation state. The implementation includes a pointing tip rotatable coupled extensible arm 1002, comprising an extendible segment 1004 slidably coupled to a fixed segment 1006. The extendible segment includes a pointing tip 1008. FIG. 10b shows a side view on the FIG. 10a projection plane 10b-10b.

### Example Six—Pivoting Arm, Pointer Type Guide, Rotation Position Grooves

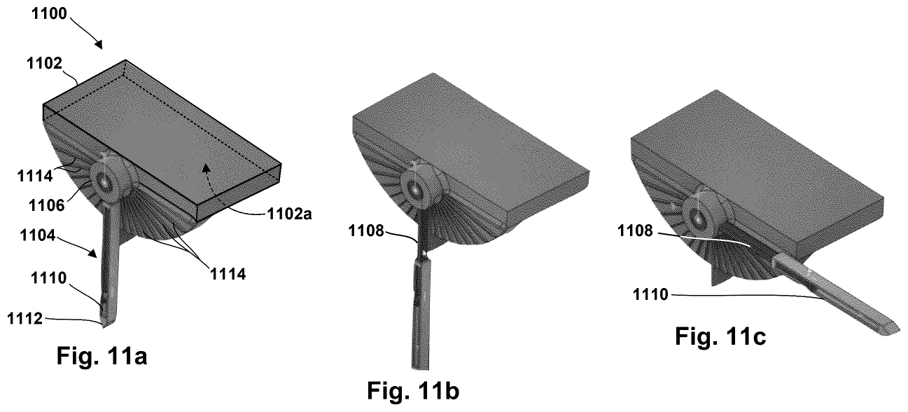

FIG. 11a shows a 3D perspective view of one alternative extendible support arm structured root tip surgical guide device 1100 according to one or more embodiments. The root tip surgical guide device 1100 comprises a bite block 1102, an extendible arm 1104 rotatably coupled to the bite block 1002 by a rotatable coupler 1106, and a locking groove protractor support 1108 that can be cooperatively arranged with the extendible arm 1104. The device uses a locking grooves mechanism to secure both the angle and length settings accurately. This ensures stable positioning during use and prevents any unintentional changes. The base can feature raised notches representing angles, for example, from 0° to 180°. Each notch corresponds to an angular increment ranging from 10° to 15°. The extendible arm can comprise an internal cavity. After selecting the desired angle, the internal cavity of the extendable arm can be inserted into the corresponding notch to lock in the angle. The extending arm can comprise a rotatably movable component that can be connected to the protractor base. The rotatably movable components can include a rear hollow section that can fit into the appropriate notch, allowing the arm to be fixed at a specific angle. The arm can be extendable to simulate the actual root length based on radiographic data. The arm or a proximal structure, or both, can be equipped with longitudinal markings, e.g., a small built-in ruler, to measure and lock in the exact length. FIG. 11b shows a 3D perspective view of the FIG. 11a alternative extendible support arm structure in another example extension state while, concurrently, supported in the FIG. 11a cooperative engagement rotation state by the multiple discrete angulation protractor support.

FIG. 11c shows a 3D perspective view of the FIG. 11a alternative extendible support arm structure in the FIG. 11b example extension state while, concurrently, supported in another cooperative engagement rotation state by the multiple discrete angulation protractor support.

### Alternative Structures and Procedures

Example above are described in reference to CBCT. This scanning technique may be preferable for many applications, scenarios, and practices, but is not intended as any limitation. Alternatives to CBCT may be used in obtaining scan information having, for example, information as described in reference to FIGS. 4 and 5. Alternatives an include periapical radiographs, panoramic radiography, digital registration methods (DRM), and/or magnetic resonance imaging (MRI).

Another example structure can include a device in accordance with FIGS. 1a-1c or FIGS. 11a-11c, but substituting the extendible arm with a set of different length arms for example but without limitation integer 6 or integer 8. After obtaining the scan root tip information, the appropriate length arm can be selected, and attached, e.g., via a threaded crew or other mechanism, to a rotatable coupler. The rotatable couple can then be set in accordance with scan-obtained angulation.

Another example pertains to methods for using the root tip guide device. One operation can include inserting a settable bite impression putty between the first bite surface and the occlusal surfaces of the subject's subject teeth. This can increase comfort and stability of the device.

### Example Benefits and Advantages

Effective Use of CBCT Data: can translate CBCT-derived measurements, e.g., root length and angulation, into a configured root tip guide, i.e., into a practical, real-time surgical guidance.
Bone Preservation: can provide accurate determination of the osteotomy site, which in turn can minimize or at least substantially reduce unnecessary bone removal.
Anatomical Safety: can reduce the risk of damaging critical anatomical structures (e.g., IAN, sinus).
Time & Cost Efficiency: Can eliminate, or at least substantially reduce the need for patient-specific 3D-printed guides or custom-designed trephine burs; e.g., by utilizing above-described simple adjustable ruler and protractor system.
Educational Tool: Can increase learning efficiency for dental students and less experienced clinicians by guiding precise osteotomy localization.
No Specialized Equipment Required: Compatible with standard burs and handpieces—no need for costly tools like piezoelectric instruments.
User-Friendly: Simple to apply, adjust, and remove during surgery. Also, as will be understood by persons having ordinary skill in the arts (PHOSITAs) pertinent to this disclosure, from reading said description, the only exertion a patient need make to enable such positioning and alignment is to open the jaws enough for spacing between the occlusal surfaces of the patient's mandibular and maxillary molars plate to exceed the plate thickness.
Sterilizable: Can be constructed from stainless steel and can be safely autoclaved.
Time-Saving in Surgery: Can shorten time that is spent on intraoperative decision-making regarding the osteotomy site and angulation.

It is to be understood that practices that are within the scope of the appended claims are not limited to particular embodiments described, as embodiments described herein are susceptible to various modifications and alternative forms. Embodiments disclosed by way of example in the drawings and are described in detail herein. The example embodiments described, though, are not limited to the particular explicit implementations, configurations, arrangements, and forms disclosed. Rather, the instant disclosure supports all modifications, equivalents, and alternatives that fall within the scope of the appended claims.

It is also to be understood that the terminology used herein is for the purpose of describing particular embodiments only, and is not intended to be limiting, as the scope of exclusivity will be limited only by the appended claims.

Where a range of values is provided, it is understood that each intervening value, to the tenth of the unit of the lower limit unless the context clearly indicates or dictates otherwise, between the upper limit and lower limit of that range and any other stated or intervening value in that stated range, is encompassed within the range. The upper and lower limits of these smaller ranges may independently be included in and encompassed in the smaller ranges, subject to any specifically excluded limit in the stated range. Where the stated range includes one or both of the limits, ranges excluding either or both of those included limits are also included.

Ordinal terms such as “first,” “second,” “third,” etc., as used in this specification and the appended claims to modify, e.g., step(s), functional block(s), signal(s), instruction(s), element(s) are to be understood as a labelling to individually reference separate step(s), functional block(s), signal(s), instruction(s), and/or element(s), and are not to be understood, except where expressly stated or clearly indicated as otherwise, to be any indication of any ordering of the step(s), functional block(s), signal(s), instruction(s), and/or element(s), in terms of time, spatial arrangement, priority, or to be any indication of the presence or inclusion of any of ordinal term referenced step(s), functional block(s), signal(s), instruction(s), and/or element(s) being conditional on the presence or inclusion of any different ordinal term referenced step(s), functional block(s), signal(s), instruction(s), and/or element(s) indication of relative priority.

Unless defined otherwise, all technical and scientific terms used herein have the same meaning as commonly understood by one of ordinary skill in the art(s) to which this disclosure relates.

Representative and illustrative methods and materials are described herein. Methods and materials similar or equivalent to those described herein can also be used in practices in accordance with disclosed embodiments.

It is to be understood that as used herein and in the appended claims the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure in its entirety, each of the individual embodiments described and illustrated herein has discrete components and features that may be readily separated from the embodiment or combined with features of any of the other several embodiments without departing from the scope or spirit of the present invention.

It is to be understood that various methods and operations are described as a plurality of separate actions, steps, or operations but it will also be understood that such description can be for further assisting the reader in gaining an understanding of, for example, concepts and/or logic relationships and should not be understood as limiting the order in which the actions, steps, or operations can be performed and that these can be carried out in the temporal order or spatial arrangement recited or in any other order or arrangement that is logically possible.

While the disclosure describes features and aspects in terms of its several exemplary embodiments, those skilled in the art will recognize that the invention can be practiced with modification within the spirit and scope of the appended claims. Accordingly, the appended claims are not limited to the embodiments as described above, but should further include all modifications and equivalents thereof within the spirit and scope of the description provided herein.

This project was financially supported by the Deanship of Scientific Research at King Abdulaziz University, Jeddah, under Grant No. IFPP-017-165-2022. Technical support was provided by the Innovation and Entrepreneurship Center at King Abdulaziz University.

We claim:

1. A visible target position reference device for endodontic surgery, comprising
a support block, having an external shape geometry that includes a first biting surface that extends in a plane and a second biting surface arranged opposite the first biting surface, the external shape geometry being further configured such that the support block is manually placeable into and positionable within a human oral cavity into an alignment with a subject tooth comprising the first biting surface extending parallel to the occlusal plane of and contacting the occlusal surface of the subject tooth;
a selectively positionable visible guide member comprising:
a pivot coupler having a first pivot member that is fixed to the support block in alignment with a pivot axis, and a second pivot member that is engaged with the first pivot member at a selectable rotation angle about the pivot axis,
a support arm, having a coupling end fixed to the second pivot member, and
a visible target position indicator spaced a distance along a displacement axis from the coupling end, wherein the displacement axis intersects the reference plane at an angle, wherein the pivot coupler is further configured such that the angle is selectable.

2. The visible target position reference device of claim 1 wherein the pivot coupler comprises:
a pivot coupling first element that is secured to a side of the support block; and
a pivot coupling second element that is secured to the coupling end of the support arm and is in a rotatable engagement with the pivot coupling first element,
wherein:
the pivot coupling first element is aligned to a pivot axis,
the pivot axis extends parallel to the plane of the first biting surface,
the rotatable engagement is configured to support rotational movement of the pivot coupling second element with respect to the pivot coupling first element, about the pivot axis, between at least a first rotational position and a second rotational position.

3. The visible target position reference device of claim 2 wherein the rotational movement about the pivot axis, between at least the first rotational position and the second rotational position correspondingly moves the visible targe position indicator along an arcuate path, which is centered at the pivot axis and extends in a movement plane that is normal to the pivot axis.

4. The visible target position reference device of claim 2, further comprising a support structure, attached to the support block, configured to support the support arm against flexion that displaces the visible target position indicator in a direction toward a facing gingiva surface of the patient.

5. The visible target position reference device of claim 2, further comprising a selectively securable rotation structure, configured to have:
a released state wherein the arm is movable such that the visible target position indicator is movable along an arcuate path, and
a fixed state wherein the visible target position indicator is fixed.

6. The visible target position reference device of claim 4, wherein
the selectively positionable visible guide member further comprises a multiple slotted protractor element that comprises at least a first angle securement slot and a second angle securement slot, and
the support arm includes a slot engagement surface.

7. The visible target position reference device of claim 2, wherein the support arm comprises a selectively extendable arm configured such that the displacement distance is selectable.

8. The surgery guide apparatus of claim 1, wherein the visible target position indicator comprises a guide structure that includes a guide ring that is centered on the target position.

9. A method for guided endodontic root tip surgery:
applying a cone-beam computed tomography (CBCT) measuring to a patient, outputting a root tip length data and a root angulation data,
based at least in part on the root tip length data and the root tip angulation data, constructing a configured visible target position reference device according to claim 1, wherein the angle with the reference plane is based at least in part on the root tip angulation data.

10. A method for fabricating a guide apparatus, comprising:
receiving an anatomical measurement data for a subject patient, wherein the anatomical measurement data includes a root angulation data and a root length data;
receiving a bite plate, having a visible target position indicator and having a shape geometry that includes a first occlusal force reception surface that generally aligns to a reference plane and, and a second occlusal force reception surface that is configured to face generally opposite the first occlusal force reception surface, wherein the bite plate is configured for manual insertion and removal from a human oral cavity and for bite force securing between opposing occlusal surfaces within the oral cavity, wherein selectively positionable visible guide member comprises:
a support arm, having a coupling end and, at the coupling end, a coupling mechanism that is configured to couple the coupling end to the bite plate,
a visible target position indicator spaced a distance along a displacement axis from the coupling end, wherein the displacement axis intersects the reference plane at an angle,
wherein the coupling mechanism is further configured such that the angle with the reference plane is selectable, and
selecting the angle of rotation and the arm extension based on the measurement data.
